(19) United States
(12) Patent Application Publication
(10) Pub. No.: US 2025/0064511 A1
(43) Pub. Date: Feb. 27, 2025

(54) METHODS AND APPARATUS FOR MULTI-CATHETER TISSUE ABLATION

(71) Applicant: Boston Scientific Scimed, Inc., Maple Grove, MN (US)

(72) Inventor: Gary L. Long, Cincinnati, OH (US)

(21) Appl. No.: 18/943,623

(22) Filed: Nov. 11, 2024

Related U.S. Application Data
(63) Continuation of application No. 17/207,053, filed on Mar. 19, 2021, now Pat. No. 12,137,968.
Application No. 17/207,053 is a continuation of application No. 15/795,075, filed on Oct. 26, 2017, now abandoned.
Application No. 15/795,075 is a continuation of application No. 15/341,523, filed on Nov. 2, 2016, now abandoned.
(63) Continuation of application No. PCT/US2015/031086, filed on May 15, 2015.
(60) Provisional application No. 61/996,855, filed on May 16, 2014.

(51) Int. Cl. A61B 18/14; A61B 17/00; A61B 18/00; A61B 18/12

(52) U.S. Cl. CPC A61B 18/1492; A61B 2017/00154; A61B 2018/00363; A61B 2018/00577; A61B 2018/00613; A61B 2018/00654; A61B 2018/00708; A61B 2018/00839; A61B 2018/00875; A61B 18/1206; A61B 2018/124; A61B 2018/126; A61B 2018/1266

(57) ABSTRACT

Catheter systems, tools and methods are disclosed for the selective and rapid application of DC voltage to drive irreversible electroporation, with the system controller configurable to apply voltages to an independently selected subsets of electrodes, such that voltages of one polarity are applied to a multiplicity of electrodes on a first medical device and voltages of the opposite polarity to a multiplicity of electrodes on a second medical device. The first and second medical devices can be epicardial catheters positioned such that their opposing distal tips are approximately aligned and whose segments with electrodes collectively wrap around the pulmonary veins.

### CROSS-REFERENCE TO RELATED APPLICATIONS

The present application is a continuation of U.S. patent application Ser. No. 17/207,053, filed Mar. 19, 2021, which is a continuation of U.S. patent application Ser. No. 15/795,075, filed Oct. 26, 2017, which is a continuation of U.S. patent application Ser. No. 15/341,523, filed Nov. 2, 2016, which is a continuation of PCT Application No. PCT/US2015/031086, filed May 15, 2015, which claims priority to U.S. Provisional Application Ser. No. 61/996,855, filed May 16, 2014, the entire disclosures of which are incorporated herein by reference.

### BACKGROUND

The embodiments described herein relate generally to medical devices for therapeutic electrical energy delivery, and more particularly to systems and methods for delivering electrical energy in the context of ablating tissue rapidly and selectively by the application of suitably timed pulsed voltages that generate irreversible electroporation of cell membranes.

The past two decades have seen advances in the technique of electroporation as it has progressed from the laboratory to clinical applications. Known methods include applying brief, high voltage DC pulses to tissue, thereby generating locally high electric fields, typically in the range of hundreds of Volts/centimeter. The electric fields disrupt cell membranes by generating pores in the cell membrane, which subsequently destroys the cell membrane and the cell. While the precise mechanism of this electrically-driven pore generation (or electroporation) awaits a detailed understanding, it is thought that the application of relatively large electric fields generates instabilities in the phospholipid bilayers in cell membranes, as well as mitochondria, causing the occurrence of a distribution of local gaps or pores in the membrane. If the applied electric field at the membrane exceeds a threshold value, typically dependent on cell size, the electroporation, is irreversible and the pores remain open, permitting exchange of material across the membrane and leading to apoptosis or cell death. Subsequently, the surrounding tissue heals in a natural process.

While pulsed DC voltages are known to drive electroporation under the right circumstances, the examples of electroporation applications in medicine and delivery methods described in the prior art do not discuss specificity of how electrodes are selected to accomplish the desired ablation. For example, some known catheters and systems include a single multi-electrode catheter in which certain electrodes receive a voltage signal having a first polarity and other electrodes receive a voltage signal having the opposite polarity. Accordingly, to minimize the risk of dielectric breakdown within the catheter, such known catheters typically include substantial insulation (e.g., around the leads), thus increasing the size and limiting the flexibility of the catheter. Some known catheters configured to produce voltage pulses of up to 5 kV include catheter leads having an insulation of as much as 0.2 mm and an overall size of about 14 French (4.67 mm).

There is a need for selective energy delivery for electroporation and its modulation in various tissue types, as well as pulses that permit rapid action and completion of therapy delivery. This need includes methods and apparatus for placement and therapy delivery from the same device or a set of devices, especially in the context of ablation therapy for cardiac arrhythmias with epicardial catheter devices. There is a need for thin, flexible, atraumatic devices that can, at the same time, effectively deliver high DC voltage electroporation ablation therapy selectively to tissue in regions of interest. Such more selective and effective electroporation delivery methods can broaden the areas of clinical application of electroporation including therapeutic treatment of a variety of cardiac arrhythmias.

### SUMMARY

Catheter systems, tools and methods are disclosed for the selective and rapid application of DC voltage to drive electroporation. In some embodiments, an apparatus includes a voltage pulse generator and an electrode controller. The voltage pulse generator is configured to produce a pulsed voltage waveform. The electrode controller is configured to be operably coupled to the voltage pulse generator. The electrode controller includes a first output port and a second output port. The first output port is configured to be operatively coupled to a first medical device including a first set of electrodes, and the second output port is configured to be operatively coupled to a second medical device including a second set of electrodes. The electrode controller includes a selection module and a pulse delivery module. The selection module is configured to select at least a first electrode from the first set of electrodes and identify at least the first electrode as an anode. The selection module is configured to select at least a second electrode from the second set of electrodes and identify at least the second electrode as a cathode. The pulse delivery module is configured to deliver a first output signal having a first polarity and being associated with the pulsed voltage waveform to the first output port for application to the first electrode. The pulse delivery module is configured to deliver a second output signal having a second polarity opposite the first polarity and being associated with the pulsed voltage waveform to the second output port for application to the second electrode.

### BRIEF DESCRIPTION OF THE DRAWINGS

FIG. 1 is a perspective view showing two catheters according to an embodiment, each with multiple electrodes disposed along its shaft and wrapped around a portion of the pulmonary veins and being within the epicardial space of the heart in a subject body such that they form an approximately closed contour around the pulmonary veins.

FIG. 2 is a schematic illustration of a catheter according to an embodiment, with a multiplicity of electrodes disposed along its shaft, with an electrical lead attached to the inner side of each electrode, and with a magnet located near the distal end of the catheter.

FIG. 3 illustrates two flexible catheter devices with multiple electrodes disposed along their shafts and positioned to wrap around respective approximate halves of a single closed contour around the pulmonary veins in the epicardial space of the heart, with their distal ends positioned in close proximity.

FIG. 4 is an illustration of two flexible catheter devices with multiple electrodes disposed along their shafts and positioned to wrap around respective approximate halves of a single closed contour around the pulmonary veins in the epicardial space of the heart, with active electrodes identified according to an embodiment.

FIG. 5A is a schematic illustration of an irreversible electroporation system according to an embodiment that includes a voltage/signal generator, a controller capable of being configured to apply voltages to selected subsets of electrodes with independent subset selections for anode electrodes on one medical device and cathode electrodes on a second medical device.

FIG. 5B is a schematic illustration of an irreversible electroporation system according to an embodiment that includes a voltage/signal generator, a controller capable of being configured to apply voltages to selected subsets of electrodes with independent subset selections for anode electrodes on one medical device and cathode electrodes on a second medical device.

FIG. 6 is an illustration of an ECG waveform showing the refractory periods during atrial and ventricular pacing, and the time windows for irreversible electroporation ablation.

FIG. 7 is a schematic illustration of a method of selecting subsets of electrodes on two catheters as anodes or cathodes, according to an embodiment, whereby the ablation vector or predominant current density direction vector is selected for ablation.

FIG. 8 is a schematic illustration of a user interface of the present invention, showing electrodes on two catheters, and buttons for selection or marking of anode electrode subsets and cathode electrode subsets.

FIG. 9 is a schematic illustration of a user interface according to an embodiment, for selection of anode and cathode electrode subsets, showing a single selected anode electrode on one catheter and two selected cathode electrodes on a second catheter.

FIG. 10 is a schematic illustration of a waveform generated by the irreversible electroporation system according to an embodiment, showing a balanced square wave.

FIG. 11 is a schematic illustration of a waveform generated by the irreversible electroporation system according to an embodiment, showing a balanced biphasic square wave.

FIG. 12 is a schematic illustration of a waveform generated by the irreversible electroporation system according to an embodiment, showing a progressive balanced biphasic square wave.

### DETAILED DESCRIPTION

Systems and methods are disclosed for the selective and rapid application of DC voltage to drive electroporation. In some embodiments, an irreversible electroporation system includes a DC voltage/signal generator and a controller capable of being configured to apply voltages to a selected multiplicity or a subset of electrodes, with anode and cathode subsets being selected independently on distinct medical devices. The controller is additionally capable of applying control inputs whereby selected pairs of anode-cathode subsets of electrodes can be sequentially updated based on a pre-determined sequence.

In some embodiments, an irreversible electroporation system includes a DC voltage/signal generator and a controller capable of being configured to apply voltages to a selected multiplicity or a subset of electrodes, with independent subset selections for anode and cathode electrode selections on distinct catheter devices respectively. Further, the controller is capable of applying control inputs whereby selected pairs of anode-cathode subsets of electrodes can be sequentially updated based on a pre-determined sequence. The generator can output waveforms that can be selected to generate a sequence of voltage pulses in either monophasic or biphasic forms and with either constant or progressively changing amplitudes.

In some embodiments, an apparatus includes a voltage pulse generator and an electrode controller. The voltage pulse generator is configured to produce a pulsed voltage waveform. The electrode controller is configured to be operably coupled to the voltage pulse generator. The electrode controller includes a first output port and a second output port. The first output port is configured to be operatively coupled to a first medical device including a first set of electrodes, and the second output port is configured to be operatively coupled to a second medical device including a second set of electrodes. The electrode controller includes a selection module and a pulse delivery module. The selection module is configured to select at least a first electrode from the first set of electrodes and identify at least the first electrode as an, anode. The selection module is configured to select at least a second electrode from the second set of electrodes and identify at least the second electrode as a cathode. The pulse delivery module is configured to deliver a first output signal having a first polarity and being associated with the pulsed voltage waveform to the first output port for application to the first electrode. The pulse delivery module is configured to deliver a second output signal having a second polarity opposite the first polarity and being associated with the pulsed voltage waveform to the second output port for application to the second electrode.

In some embodiments, an apparatus includes a voltage pulse generator and an electrode controller. The voltage pulse generator is configured to produce a pulsed voltage waveform. The electrode controller is configured to be operably coupled to the voltage pulse generator. The electrode controller includes a first output port and a second output port, the first output port configured to be operatively coupled to a first medical device including a first set of electrodes, the second output port configured to be operatively coupled to a second medical device including a second set of electrodes. The electrode controller includes a selection module and a pulse delivery module. The selection module is configured to select a set of anode cathode pairs, each anode selected being only in the first plurality of electrodes, each cathode selected being only in the second plurality of electrodes. The pulse delivery module is configured to deliver a first output signal having a first polarity and associated with the pulsed voltage waveform to the first output port for application to each anode selected. The pulse delivery module is configured to deliver a second output signal having a second polarity opposite the first polarity and associated with the pulsed voltage waveform to the second output port for application to each cathode selected. The pulse delivery module is configured to deliver the first output signal and the second output signal to the plurality of anode/cathode pairs according to a sequential pattern.

Methods of control and DC voltage application from a generator capable of selective excitation of sets of electrodes are also disclosed herein. In some embodiments, a method includes identifying, via a selection module of an electrode controller, a set of anode cathode pairs, each anode selected being only in a first set of electrodes of a first multi-electrode catheter, each cathode selected being only in a second set of electrodes of a second multi-electrode catheter. The first multi-electrode catheter and the second multi-electrode catheter are configured to collectively surround a portion of a heart. A pacing signal is conveyed to a pacing lead configured to be operatively coupled to the heart. The method includes receiving, at a feedback module, an electrocardiogramal associated with a function of the heart. The method includes delivering, via a pulse delivery module of the electrode controller, a first output signal having a first polarity to each anode selected, and delivering, via the pulse delivery module, a second output signal having a second polarity opposite the first polarity to each cathode selected. The first output signal and the second output signal are delivered according to a sequential pattern.

In some embodiments, a non-transitory processor readable medium storing code representing instructions to be executed by a processor includes code to cause the processor to identify a set of anode/cathode pairs. Each anode in the plurality of anode/cathode pairs being only in a first set of electrodes of a first multi-electrode catheter. Each cathode in the set of anode/cathode pairs being only in a second set of electrodes of a second multi-electrode catheter. The first multi-electrode catheter and the second multi-electrode catheter are configured to collectively surround a portion of a heart. The code further includes code to convey a pacing signal to a pacing lead configured to be operatively coupled to the heart, and receive an electrocardiogramal associated with a function of the heart. The code further includes code to deliver, according to a sequential pattern, a first output signal having a first polarity to each anode selected and a second output signal having a second polarity opposite the first polarity to each cathode selected.

In some embodiments system includes a first flexible catheter including a first set of electrodes and a second flexible catheter including a second set of electrodes. A distal end portion of the first flexible catheter is configured to be coupled to a distal end portion of the second catheter to form a continuous length including the first set of electrodes and the second set of electrodes. The first flexible catheter and the second flexible catheter are configured to deliver a bipolar voltage signal to a target tissue such that a first portion of the bipolar voltage signal having a first polarity is delivered only to the first set of electrodes and a second portion of the bipolar voltage signal having second polarity opposite the first polarity is delivered only to the second set of electrodes.

As used in this specification, the singular forms “a,” “an” and “the” include plural referents unless the context clearly dictates otherwise. Thus, for example, the term “a member” is intended to mean a single member or a combination of members, “a material” is intended to mean one or more materials, “a processor” is intended to mean a single processor or multiple processors; and “memory” is intended to mean one or more memories, or a combination thereof.

As used herein, the terms “about” and “approximately” generally mean plus or minus 10% of the value stated. For example, about 0.5 would include 0.45 and 0.5.5., about 10 would include 9 to 11, about 1000 would include 900 to 1100.

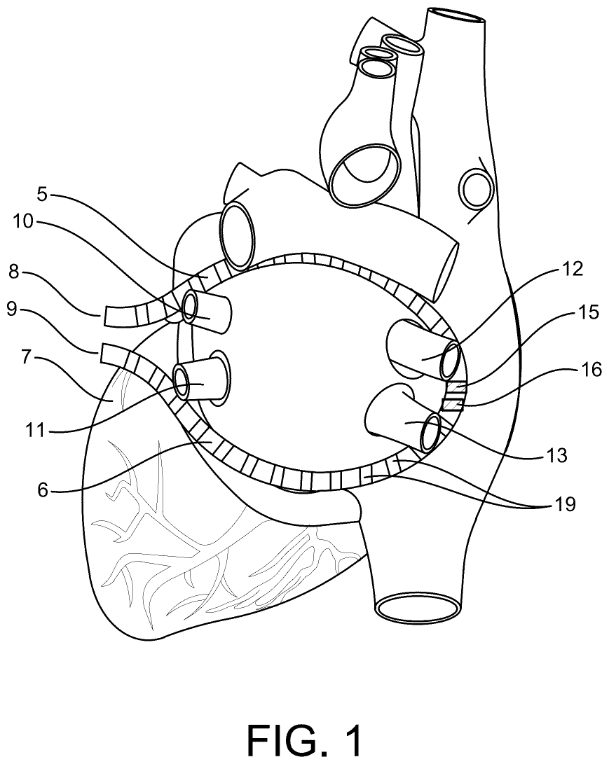

As shown in FIG. 1, in some embodiments a Pulmonary Vein isolation (PV isolation) system includes two ablation catheter devices, one (labeled 5) with distal end 15 and proximal portion 8, the other (labeled 6) with distal end 16 and proximal portion 9, each with a multiplicity of electrodes (indicated by dark bands such as those marked as 19) disposed along its length, and where each catheter is wrapped in the epicardial space around a portion of the pulmonary veins 10, 11, 12 and 13 of a heart 7 in a subject or patient anatomy, with the proximal portions 8 and 9 of the respective catheters 5 and 6 extending out and away to eventually emerge from the patient's chest. In some embodiments, the distal ends of the two catheters 5 and 6 have magnets 15 and 16 respectively that can aid in alignment of the two catheters. The ablation catheters 5 and 6, and any of the catheters described herein can be similar to the ablation catheters described in PCT Publication No. WO2014/025394, entitled “Catheters. Catheter Systems, and Methods for Puncturing Through a Tissue Structure,” filed on Mar. 14, 2013 (“the '394 PCT Application), which is incorporated herein by reference in its entirety. The ablation catheters 5 and 6 can be disposed about the pulmonary veins 10, 11, 12 and 13 using any suitable procedure and apparatus. For example, in some embodiments, the ablation catheters can be disposed about the pulmonary veins 10, 11, 12 and 13 and/or the heart 7 using a puncturing apparatus disposed via a subxiphoid pericardial access location and a using guidewire-based delivery method as described in the '394 PCT Application. Similar methods can be used to deliver and position the two catheters 5 and 6. After the ends 8 and 9 of the two respective catheters 5 and 6 extend and emerge out of the patient chest they can be cinched together to effectively hold the catheters in place or in stable positions relative to each other.

A DC voltage for electroporation can be applied to subsets of electrodes identified as anodes and cathodes respectively on the two catheters on approximately opposite sides of the closed contour defined by the shapes of the catheters 5 and 6 around the pulmonary veins. The DC voltage is applied in brief pulses sufficient to cause irreversible electroporation and can be in the range of 0.5 kV to 10 kV and more preferably in the range 1 kV to 2.5 kV, so that a threshold electric field value of around 200 Volts/cm is effectively achieved in the cardiac tissue to be ablated. In some embodiments, the marked or active electrodes on the two catheters can be automatically identified, or manually identified by suitable marking, on an X-ray or fluoroscopic image obtained at an appropriate angulation that permits identification of the geometric distance between anode and cathode electrodes, or their respective centroids. In one embodiment, the DC voltage generator setting for irreversible electroporation is then automatically identified by the electroporation system based on this distance measure. In an alternate embodiment, the DC voltage value is selected directly by a user from a suitable dial, slider, touch screen, or any other user interface. The DC voltage pulse results in a current flowing between the anode and cathode electrodes on opposite sides of the contour defined by the conjoint shapes of the two catheters, with said current flowing through the cardiac wall tissue and through the intervening blood in the cardiac chamber, with the current entering the cardiac tissue from the anode electrodes and returning back through the cathode electrodes. The forward and return current paths (leads) are respectively inside distinct catheters, since all active electrodes on a given catheter are of like polarity. Areas of cardiac wall tissue where the electric field is sufficiently large for irreversible electroporation are ablated during the DC voltage pulse application.

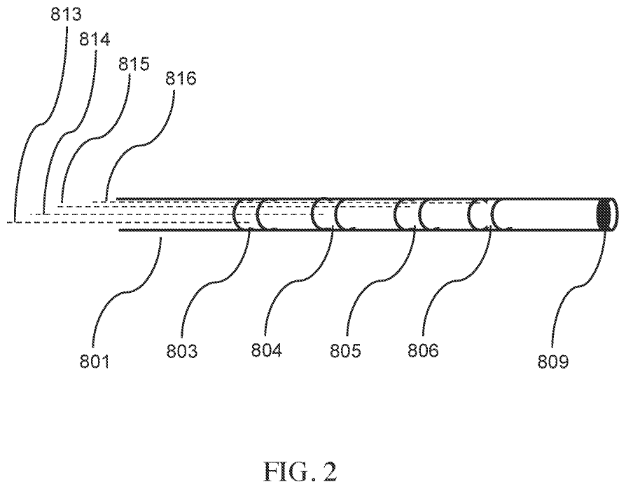

FIG. 2 is a schematic illustration of a multi-electrode, magnet-tipped catheter according to an embodiment. The catheter shaft 801 has a multiplicity of electrodes disposed along an extensive length of catheter at least 5 cm in extent. In some embodiments the metallic electrodes could be poly-metallic in construction, for example, including regions of Titanium and regions of Platinum. Although FIG. 2 shows only four electrodes 803, 804, 805 and 806 for clarity, in other embodiments, the number of electrodes can be in the range between 5 and 30, and more preferably in the range between 8 and 18. Each electrode attaches to a corresponding lead, thus as shown in FIG. 2, electrodes 803, 804, 805 and 806 attach to leads 813, 814, 815 and 816, respectively.

The catheter shaft is made of a flexible polymeric material such as for example Teflon, Nylon or Pebax. Moreover, the leads 813, 814, 815 and 816 include an insulative covering to ensure that each lead/electrode is electrically isolated from the other leads and electrodes coupled to the catheter shaft 801. When the catheter device is used with the systems and methods described herein, all of the electrodes 803, 804, 805 and 806 of the catheter 801 have the same polarity. Thus, the need for high dielectric strength material separating the leads is not a significant constraint. Accordingly, the insulative material covering each lead can be minimized, and the catheter can be relatively small in diameter. In this manner, the catheter device can have a high degree of flexibility to facilitate the method of surrounding the pulmonary veins as described herein. In some embodiments, for example, the catheter device can have a size in the range of approximately 9 French (3 mm), 8 French (2.67 mm) or even 6 French (2 mm). In some embodiments, the electrode leads of the catheter device can have an insulation thickness of less than about 0.05 mm, less than about 0.01 m, or less than about 0.005 mm. In other embodiments, the electrode leads of the catheter can have an insulation thickness of between about 0.03 mm and about 0.06 mm.

Moreover, by maintaining the voltage for each of the electrodes 803, 804, 805 and 806 of the catheter 801 at the same polarity, higher voltage levels can be applied to the electrodes of the catheter with minimal risk of dielectric breakdown. In this manner, the catheter device 801 (and the systems and methods described herein) can enhance the efficacy of irreversible electroporation ablation. For example, in some embodiments, the voltage applied to the electrodes 803, 804, 805 and 806 can be in the range of 0.5 kV to 2.5 kV; 2.5 kV to 5 kV, and up to 10 kV and more preferably in the range 1 kV to 2.5 kV.

As shown in FIG. 2, the distal end of the catheter 801 has a ring-shaped magnet 809, with the magnet having a magnetization direction that is substantially aligned with the longitudinal axis of the catheter. The magnet 809 is configured to have a polarity to cooperate with a corresponding magnet from a second catheter to be used in conjunction with the catheter 801. For example, the magnets 15 and 16 shown respectively at the distal ends of the two catheters 5 and 6 in FIG. 1 have opposite polarities on their distal faces, so that they attract each other. This attraction can aid in approximate alignment and/or coupling of the catheters. With two distinct catheters, anode and cathode electrodes for voltage application can be selected on distinct devices in accordance with the methods described herein.

Additionally, by using two catheters to deliver a bipolar pulse, but maintaining the voltage for each of the electrodes within each catheter at the same polarity, according to the methods described herein, each of the catheters includes fewer electrodes and few leads than if a single catheter were used to surround the heart. The reduction of the number of leads and electrodes also allows for an overall reduction in the size of the catheter, improvement in the flexibility thereof, and the like.

In some embodiments, for example, a system includes a first catheter including a first set of electrodes and a second flexible catheter including a second set of electrodes. Each of the catheters can be, for example, the catheter 801. A distal end portion of the first flexible catheter is configured to be coupled to a distal end portion of the second catheter to form a continuous length including the first set of electrodes and the second set of electrodes. The connection can be via a magnetic coupling device, as shown herein. The first flexible catheter and the second flexible catheter are configured to deliver a bipolar voltage signal to a target tissue such that a first portion of the bipolar voltage signal having a first polarity is delivered only to the first set of electrodes and a second portion of the bipolar voltage signal having second polarity opposite the first polarity is delivered only to the second set of electrodes.

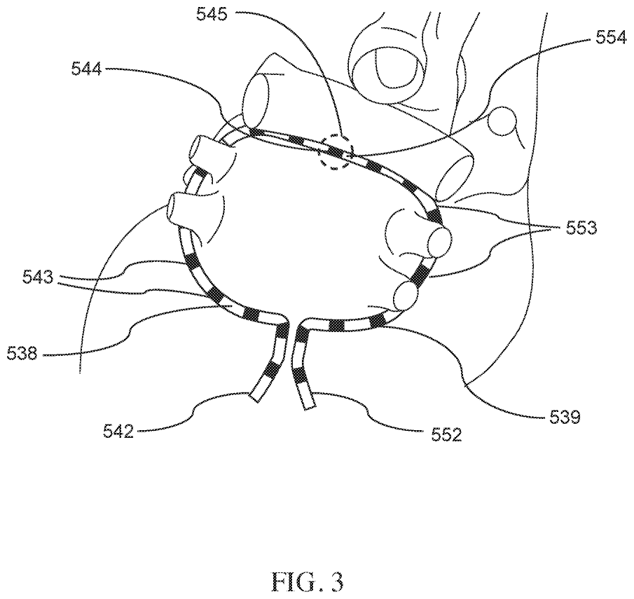

FIG. 3 shows another example of the placement of a first catheter 538 and a second catheter 539, according to an embodiment. The first catheter 538 and the second catheter 539 can each be constructed of a small diameter tube covered by multiple metal electrodes. The distal ends of the catheters (544 and 554 respectively) contain magnets that attract and allow for alignment of the devices when their distal ends are in close proximity as indicated by region 545. Electrodes 543 disposed along the first catheter 538 are of a single polarity (for instance, an activated subset of the electrodes would all be anodes), while electrodes 553 disposed along the second catheter 539 are all of the opposite polarity (in the same example, an activated subset could all be cathodes). Wires are connected to each electrode in the anode and cathode catheters, indicated by collective anode leads 542 and collective cathode leads 552 respectively. Thus, wires of opposite polarity are not in the same catheter, thereby reducing the possibility of dielectric breakdown at high voltage, as discussed above. Furthermore, when it is desired to reduce the catheter diameter and the number of leads within a given catheter, it may also be advantageous to use two distinct catheters, with the pair of catheters surround and/or substantially enclosing the pulmonary veins and with their distal ends closely abutting. Reduced diameter catheters can be significantly more flexible and a smaller number of leads in a given catheter (resulting in a smaller catheter diameter) can be advantageous in this regard. While in this example the two catheters are navigated and coupled magnetically, those skilled in the art could implement other means of alignment and positioning of the catheters without departing from the scope of the teachings herein.

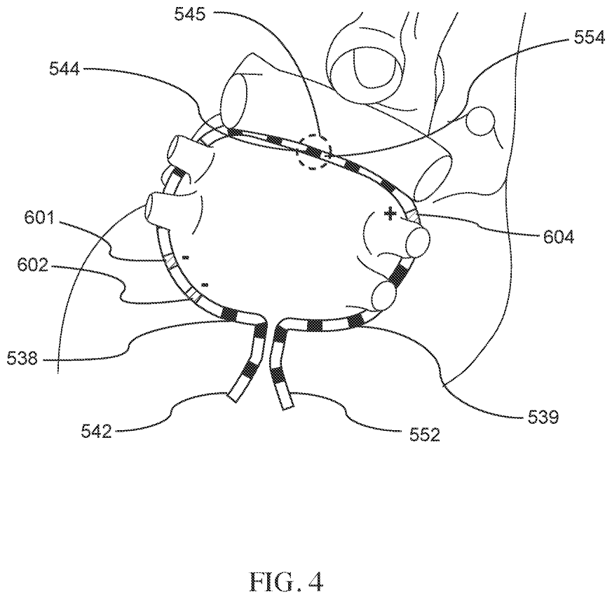

The illustration in FIG. 4 shows the first catheter 538 and the second catheter 539 having proximal leads 542 and 552, respectively, and having distal ends 544 and 554, respectively. In accordance with the systems and methods described herein, the a single active anode electrode 604 is selected on the second catheter 539, and two active cathode electrodes 601 and 602 are selected on the first catheter 538. A DC voltage for irreversible electroporation ablation can then be applied across the selected anode-cathode electrodes.

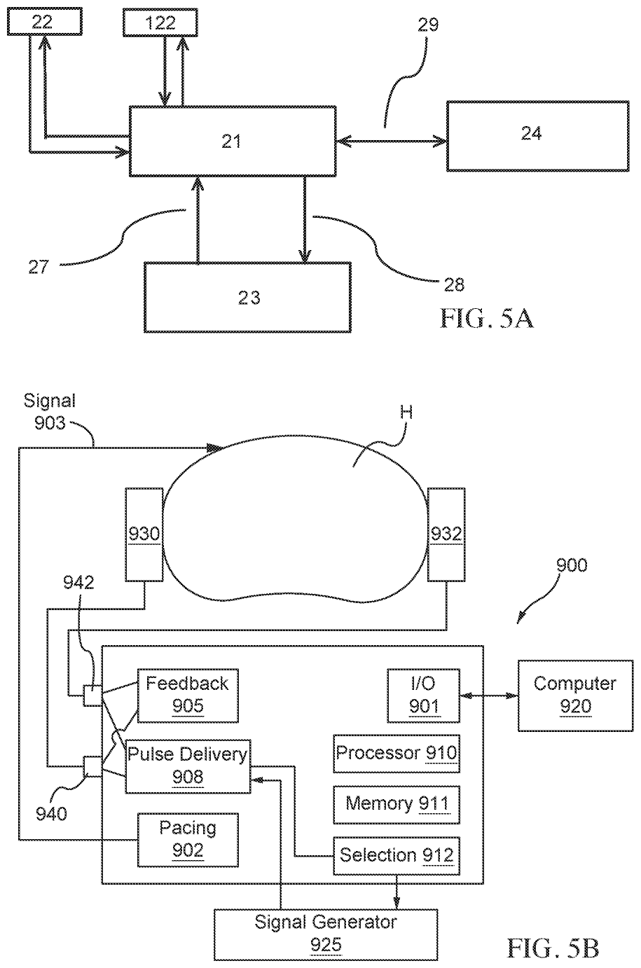

A schematic diagram of an electroporation system according to an embodiment is shown in FIG. 5A. The system includes a DC voltage/signal generator 23 that is driven by a controller unit 21. The controller unit 21 interfaces with a computer device 24 by means of a two-way communication link 29. The controller interface can act as a multiplexer unit and perform channel selection and routing functions for applying DC voltages to appropriate electrodes that have been selected by a user or by the computer 24. The controller can apply the voltages via a multiplicity of leads to a first catheter device 22, as well as a second catheter device 122. Active electrodes can be selected on a first catheter device 22 with one polarity, and likewise active electrodes can be selected on a second catheter device 122 with the opposite polarity.

In some embodiments, one or more leads from the controller 21 could also carry pacing signals to drive pacing of the heart through a separate pacing device (not shown). The catheter devices can also send back information such as ECG recordings or data from other sensors back to the controller 21, possibly on separate leads. While the DC voltage generator 23 sends a DC voltage to the controller 21 through leads 27, the voltage generator is driven by control and timing inputs 28 from the controller unit 21.

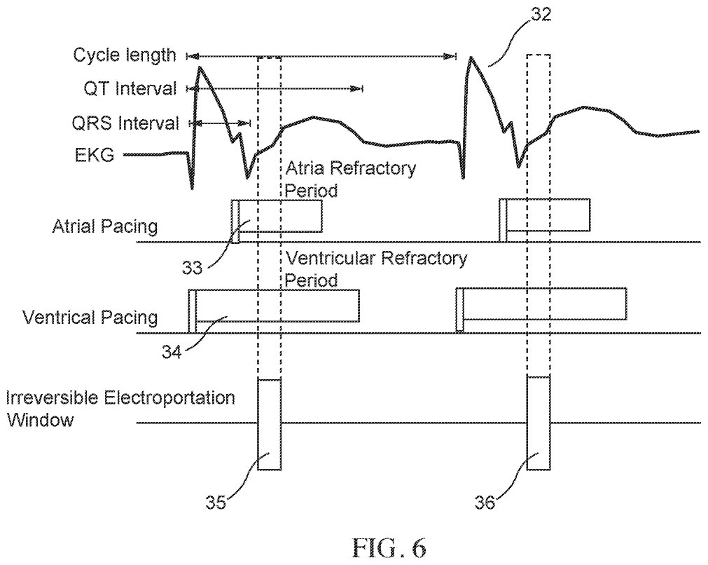

As shown in FIG. 6, given atrial or ventricular pacing inputs to the heart, the resulting ECG waveform 32 has appropriate respective refractory time intervals 33 and 34 respectively, during which there are suitable time windows for application of irreversible electroporation as indicated by 35 and 36. The application of cardiac pacing results in a periodic, well-controlled sequence of electroporation time windows. Typically, this time window is of the order of hundreds of microseconds to about a millisecond or more. During this window, multiple DC voltage pulses can be applied to ensure that sufficient tissue ablation has occurred. The user can repeat the delivery of irreversible electroporation over several successive cardiac cycles for further confidence. Thus, in some embodiments, a feedback module (e.g., feedback module 905) can receive the electrocardiogramal, and a pulse delivery module (e.g., pulse delivery module 908) can deliver the output signal to the subset of electrodes during a time window associated with at least one a pacing signal or the electrocardiogramal.
in one embodiment, the ablation controller and signal generator can be mounted on a rolling trolley, and the user can control the device using a touchscreen interface that is in the sterile field. The touchscreen can be for example an LCD touchscreen in a plastic housing mountable to a standard medical rail or post and can be used to select the electrodes for ablation and to ready the device to fire. The interface can for example be covered with a clear sterile plastic drape. The operator can select the number of electrodes involved in an automated sequence. The touch screen graphically shows the catheters that are attached to the controller. In one embodiment the operator can select electrodes from the touchscreen with appropriate graphical buttons. The operator can also select the pacing stimulus protocol (either internally generated or externally triggered) from the interface. Once pacing is enabled, and the ablation sequence is selected, the operator can initiate or verify pacing. Once the operator verifies that the heart is being paced, the ablation sequence can be initiated by holding down a hand-held trigger button that is in the sterile field. The hand-held trigger button can be illuminated red to indicate that the device is “armed” and ready to ablate. The trigger button can be compatible for use in a sterile field and when attached to the controller can be illuminated a different color, for example white. When the device is firing, the trigger button flashes in sequence with the pulse delivery, in a specific color such as red. The waveform of each delivered pulse is displayed on the touchscreen interface. A graphic representation of the pre and post impedance between electrodes involved in the sequence can also be shown on the interface, and this data can be exported for file storage.

In one embodiment, an impedance map can be generated based on voltage and current recordings across anode-cathode pairs or sets of electrodes (anodes and cathodes respectively being on distinct catheters), and an appropriate set of electrodes that are best suited for ablation delivery in a given region can be selected based on the impedance map or measurements, either manually by a user or automatically by the system. Such an impedance map can be produced, for example, by the feedback module 905, or any other suitable portion of the electrode controller 900. For example, if the impedance across an anode/cathode combination of electrodes is a relatively low value (for example, less than 25 Ohms), at a given voltage the said combination would result in relatively large currents in the tissue and power dissipation in tissue. In such circumstances, this electrode combination would then be ruled out for ablation due to safety considerations (e.g., via the selection module 912), and alternate electrode combinations would be sought by the user. In a preferred embodiment, a pre-determined range of impedance values, for example 30 Ohms to 300 Ohms, could be used as an allowed impedance range within which it is deemed safe to ablate. Thus, in some embodiments, an electrode controller can automatically determine a subset of electrodes to which voltage pulses should be applied.

The waveforms for the various electrodes can be displayed and recorded on the case monitor and simultaneously outputted to a standard connection for any electrophysiology (EP) data acquisition system. With the high voltages involved with the device, the outputs to the EP data acquisition system needs to be protected from voltage and/or current surges. The waveforms acquired internally can be used to autonomously calculate impedances between each electrode pair. The waveform amplitude, period, duty cycle, and delay can all be modified, for example via a suitable Ethernet connection. Pacing for the heart is controlled by the device and outputted to the pacing leads and a protected pacing circuit output for monitoring by a lab.

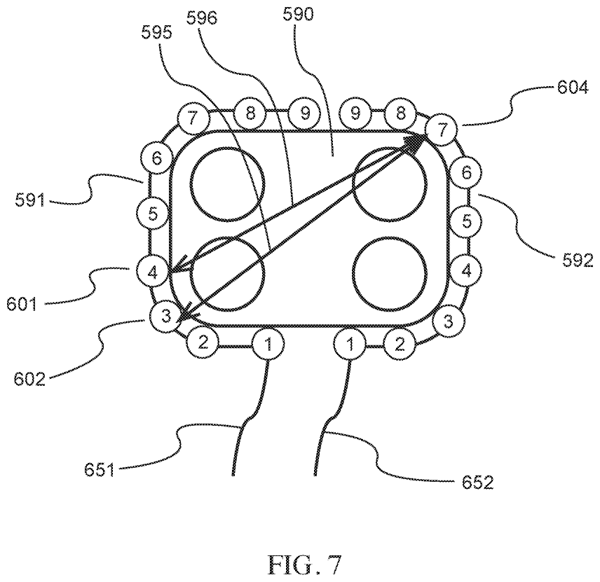

While a touchscreen interface is one preferred embodiment, other user interfaces can be used by a user to control the system such as a graphical display on a laptop or monitor display controlled by a standard computer mouse or joystick. FIG. 7 shows a schematic rendering of a portion of the user interface of the electroporation system. The graphic shown in the FIG. represents a specific choice of electrode subsets for anode and cathode selection. The two PV isolation ablation catheters in the FIG. are represented by strings of numbered electrodes as indicated respectively by 591 and 592, wrapped around the area 590 of the pulmonary veins represented by the gray region in this schematic diagram for ease of user visualization. The catheters 591 and 592 have proximal leads 651 and 652 respectively that connect to a controller or interface unit as described earlier. Referring to the figure, the arrows 595 and 596 represent approximate current density vectors, with one end at the cathodes and the other end at the anodes; in this figure, the three electrodes marked 601 and 602 are cathodes, and the single electrode marked 604 is the anode. It is apparent from FIG. 7 that the user can select various subsets of electrodes (respectively on distinct catheters) as cathode or anode, depending on the region to be ablated along the length of the contour around the pulmonary veins represented by the two catheters. In one embodiment, the user can make one selection of cathode and anode subsets, and the system can take this selection as input to generate an ablation sequence that moves around the ring or contour defined by the shapes of the two PV isolation catheters, for example moving clockwise at each step with a one-electrode displacement. In this manner, the pair of cathode and anode electrode subsets can be sequentially updated for ablation purposes, so that if there are N12 electrodes on each catheter, after N updates the entire contour has been updated such that the current arrows shown as 595 and 596 have swept once around the contour completely.

In some cases, the portion of one of the PV isolation catheters with electrodes may be longer than needed to wrap around a given patient's pulmonary veins; in this event, a smaller number of electrodes is sufficient to wrap around the contour of the pulmonary veins. These define the number of “active” electrodes to be used in the ablation process.

In a some embodiments, the system (any of the generators and controllers described herein) can deliver rectangular-wave pulses with a peak maximum voltage of about 5 kV into a load with an impedance in the range of 30 Ohm to 3000 Ohm for a maximum duration of 200 us, with a 100 us maximum duration being still more preferred. Pulses can be delivered in a multiplexed and synchronized manner to a multi-electrode catheter inside the body with a duty cycle of up to 50% (for short bursts). The pulses can generally be delivered in bursts, such as for example a sequence of between 2 and 10 pulses interrupted by pauses of between 1 ms and 1000 ms. The multiplexer controller is capable of running an automated sequence to deliver the impulses/impulse trains (from the DC voltage signal/impulse generator) to the tissue target within the body. The controller system is capable of switching between subsets/nodes of electrodes located on the single-use catheters. Further, the controller can measure voltage and current and tabulate impedances in each electrode configuration (for display, planning, and internal diagnostic analysis). It can, also generate two channels of cardiac pacing stimulus output, and is capable of synchronizing impulse delivery with the internally generated cardiac pacing and/or an external trigger signal. In one embodiment, it can provide sensing output/connection for access to bio potentials emanating from each electrode connected to the system (with connectivity characteristics being compatible with standard electrophysiological laboratory data acquisition equipment).

In some embodiments, the controller (e.g., the electrode controller 900) can automatically “recognize” each of the two single-use disposable catheters when it is connected to the controller output (prompting internal diagnostics and user interface configuration options). The controller can have at least two unique output connector ports (e.g., the first output port 940 and the second output port 942) to accommodate up to at least two catheters at once. The controller device can function as long as at least two recognized catheters are attached to it. In some embodiments, the controller can have several sequence configurations that provide the operator with at least some variety of programming options. In one configuration, the controller can switch electrode configurations of a bipolar set of electrodes (cathodes and anodes respectively on distinct catheters) sequentially in a clockwise manner (for example, starting at a given step, in the next step of the algorithm, the next cathode electrode on one catheter and the next anode electrode on the other catheter are automatically selected, timed to the synchronizing trigger), with the two catheters and their electrodes arranged in a quasi-circumference around the target. Thus in the first sequence, pulse delivery occurs so that the approximate vector of current density changes as the automated sequencing of the controller switches “on” and “off” between different electrodes surrounding the tissue target sequence. The current density vectors generally cross the target tissue but in some configurations the current density could be approximately tangential to the target. In a second sequence configuration, the impulses are delivered to user-selected electrode subsets of catheters that are connected to the device (the vector of current density does not change with, each synchronized delivery). The user can also configure the controller to deliver up to 2 channels of pacing stimulus to electrodes connected to the device output. The user can control the application of DC voltage with a single handheld switch. A sterile catheter or catheters can be connected to the voltage output of the generator via a connector cable that can be delivered to the sterile field. In one embodiment, the user activates the device with a touch screen interface (that can be protected with a single-use sterile transparent disposable cover commonly available in the catheter lab setting). The generator can remain in a standby mode until the user is ready to apply pulses at which point the user/assistant can put the generator into a ready mode via the touchscreen interface. Subsequently the user can select the sequence, the active electrodes, and the cardiac pacing parameters.

Once the catheters have been advanced to or around the cardiac target, the user can initiate electrically pacing the heart (using a pacing stimulus generated by the ablation controller or an external source synchronized to the ablation system). The operator verifies that the heart is being paced and uses the hand-held trigger button to apply the synchronized bursts of high voltage pulses. The system can continue delivering the burst pulse train with each cardiac cycle as long as the operator is holding down a suitable “fire” button or switch. During the application of the pulses, the generator output is synchronized with the heart rhythm so that short bursts are delivered at a pre-specified interval from the paced stimulus. When the train of pulses is complete, the pacing continues until the operator discontinues pacing.

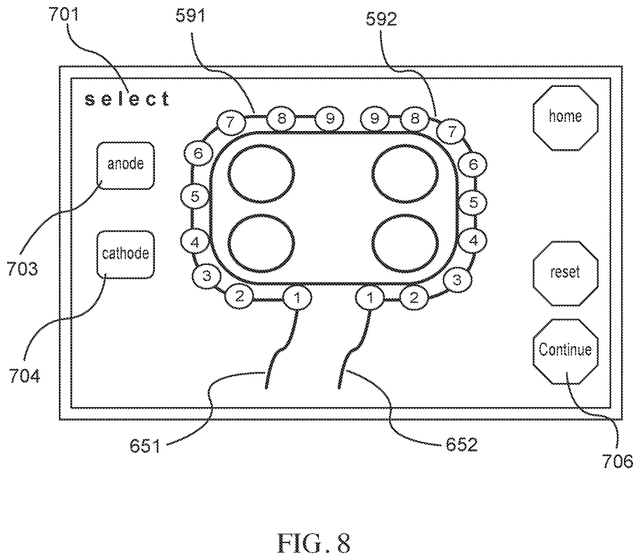

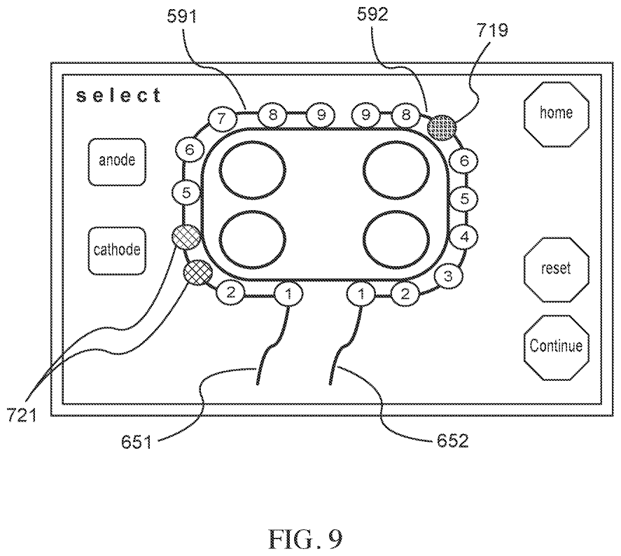

FIG. 8 shows a portion of a user interface of the electroporation system for selection (with graphical button 701) of anode and cathode electrodes, with two catheters connected to the system. One of the catheters is a PV isolation catheter 591 while the other is a PV isolation catheter 592, with their leads schematically indicated by 651 and 652 respectively. The buttons 703 and 704 can enable the selection of marked electrode subsets on the catheters as respectively anode or cathode with a “Continue” button 706. Once the selection is made, the appropriate electrodes are colored differently to indicate anode or cathode electrodes as shown marked respectively as 719 and 721 on catheters 592 and 591 respectively in FIG. 9.

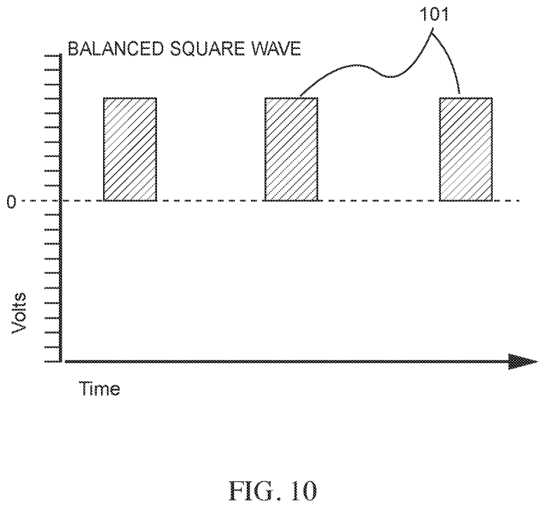

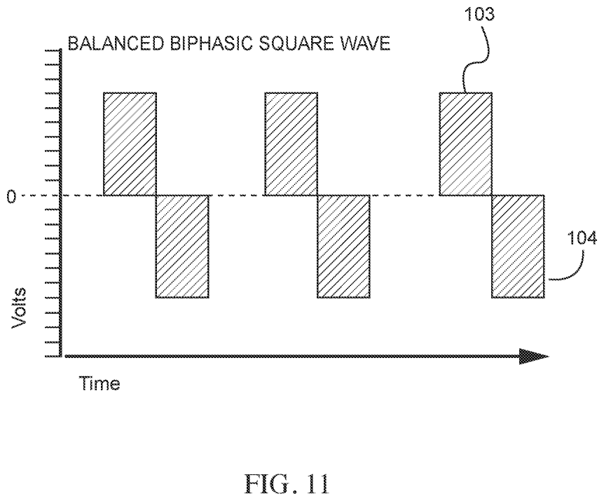

The controller and generator can output waveforms that can be selected to generate a sequence of voltage pulses in either monophasic or biphasic forms and with either constant or progressively changing amplitudes. FIG. 10 shows a rectangular wave pulse train where the pulses 101 have a uniform height or maximum voltage. FIG. 11 shows an, example of a balanced biphasic rectangular pulse train, where each positive voltage pulse such as 103 is immediately followed by a negative voltage pulse such as 104 of equal amplitude and opposite sign. While in this example the biphasic pulses are balanced with equal amplitudes of the positive and negative voltages, in other embodiments an unbalanced biphasic waveform could also be used as may be convenient for a given application.

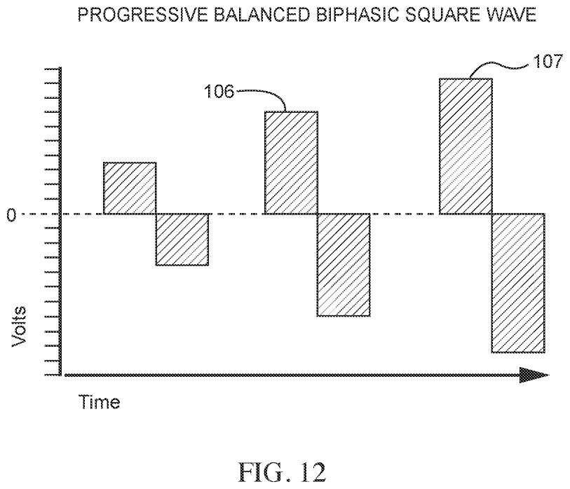

Yet another example of a waveform or pulse shape that can be generated by the system is illustrated in FIG. 12, which shows a progressive balanced rectangular pulse train, where each distinct biphasic pulse has balanced or equal-amplitude positive and negative voltages, but each pulse such as 107 is larger in amplitude than its immediate predecessor 106. Other variations such as a progressive unbalanced rectangular pulse train, or indeed a wide variety of other variations of pulse amplitude with respect to time can be conceived and implemented by those skilled in the art based on the teachings herein.

The time duration of each irreversible electroporation rectangular voltage pulse could lie in the range from 1 nanosecond to 10 milliseconds, with the range 10 microseconds to 1 millisecond being more preferable and the range 50 microseconds to 300 microseconds being still more preferable. The time interval between successive pulses of a pulse train could be in the range of 10 microseconds to 1 millisecond, with the range 50 microseconds to 300 microseconds being more preferable. The number of pulses applied in a single pulse train (with delays between individual pulses lying in the ranges just mentioned) can range from 1 to 100, with the range 1 to 10 being more preferable. As described in the foregoing, a pulse train can be driven by a user-controlled switch or button, in one embodiment preferably mounted on a hand-held joystick-like device. In one mode of operation a pulse train can be generated for every push of such a control button, while in an alternate mode of operation pulse trains can be generated repeatedly during the refractory periods of a set of successive cardiac cycles, for as long as the user-controlled switch or button is engaged by the user.

In some embodiments, a method includes identifying, via a selection module of an electrode controller, a set of anode/cathode pairs. Each anode selected in the set of anode/cathode pairs is only in a first set of electrodes of a first multi-electrode catheter. Each cathode selected in the plurality of anode/cathode pairs is only in a second set of electrodes of a second multi-electrode catheter. The first multi-electrode catheter and the second multi-electrode catheter are configured to collectively surround a portion of a heart, as described herein. In some embodiments, the identifying can be based on a predetermined schedule of electrodes. In yet other embodiments, the identifying can be performed automatically based on an impedance measurement or map as described herein.

The method further includes conveying a pacing signal to a pacing lead configured to be operatively coupled to the heart, and receiving, at a feedback module of the electrode controller, an electrocardiogramal associated with a function of the heart.

The method further includes delivering, via a pulse delivery module of the electrode controller, a first output signal having a first polarity to each anode selected. The method further includes delivering, via the pulse delivery module, a second output signal having a second polarity to each cathode selected. The first output signal and the second output signal being delivered according to a sequential pattern.

Some embodiments described herein relate to a computer storage product with a non-transitory computer-readable medium (also can be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also can be referred to as code) may be those designed and constructed for the specific purpose or purposes. Examples of non-transitory computer-readable media include, but are not limited to: magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules; and hardware devices that are specially configured to store and execute program code, such as Application-Specific Integrated Circuits (ASICs), Programmable Logic Devices (PLDs), Read-Only Memory (ROM) and Random-Access Memory (RAM) devices.

Examples of computer code include, but are not limited to, micro-code or micro-instructions, machine instructions, such as produced by a compiler, code used to produce a web service, and files containing higher-level instructions that are executed by a computer using an interpreter. For example, embodiments may be implemented using imperative programming languages (e.g., C, Fortran, etc.), functional programming languages (Haskell, Erlang, etc.), logical programming languages (e.g., Prolog), object-oriented programming languages (e.g., Java, C++, etc.) or other suitable programming languages and/or development tools. Additional examples of computer code include, but are not limited to, control signals, encrypted code, and compressed code.

While various specific examples and embodiments of systems and tools for selective tissue ablation with irreversible electroporation were described in the foregoing for illustrative and exemplary purposes, it should be clear that a wide variety of variations and alternate embodiments could be conceived or constructed by those skilled in the art based on the teachings herein. While specific methods of control and DC voltage application from a generator capable of selective excitation of sets of electrodes were disclosed, persons skilled in the art would recognize that any of a wide variety of other control or user input methods and methods of electrode subset selection etc. can be implemented without departing from the scope of the present invention. Likewise, while the foregoing described a range of specific tools or devices for more effective and selective DC voltage application for irreversible electroporation through ionic fluid irrigation and ultrasonic agitation, including insulating balloon constructions, focal ablation tools, and a basket catheter with a multiplicity of, other device constructions or variations could be implemented by one skilled in the art by employing the principles and teachings disclosed herein without departing from the scope of the present invention, in the treatment of cardiac arrhythmias, in intravascular applications, or a variety of other medical applications.

Furthermore, while the present disclosure describes specific embodiments and tools involving irrigation with saline fluids and the use of temperature to selectively ablate tissue by taking advantage of the temperature-dependence of the threshold of irreversible electroporation, it should be clear to one skilled in the art that a variety of methods and devices for steady fluid delivery, or for tissue heating through the delivery, of focused kinetic energy or electromagnetic radiation could be implemented utilizing the methods and principles taught herein without departing from the scope of the present invention. Where schematics and/or embodiments described above indicate certain

components arranged in certain orientations or positions, the arrangement of components may be modified. For example, although the controller 900 is shown as optionally including the pacing module 902, in other embodiments, the controller 900 can interface with a separate pacing module. For example, although the controller 900 is shown as optionally including the feedback module 905, in other embodiments, the controller 900 can interface with a separate feedback module. Similarly, where methods and/or events described above indicate certain events and/or procedures occurring in certain order, the ordering of certain events and/or procedures may be modified.

Although various embodiments have been described as having particular features and/or combinations of components, other embodiments are possible having a combination of any features and/or components from any of embodiments as discussed above.

What is claimed is:

1. A method of delivering pulsed ablative energy to target tissue of a heart, the method comprising:
disposing a first flexible catheter around a first portion a cardiac chamber adjacent to the target tissue, the first flexible catheter having a first distal end portion including a first magnet having a first magnetic polarity, the first flexible catheter including a first plurality of electrodes;
disposing a second flexible catheter around a second portion of the cardiac chamber adjacent to the target tissue, the second flexible catheter having a second distal end portion including a second magnet having a second magnetic polarity opposite to the first magnetic polarity, the second flexible catheter including a second plurality of electrodes;
coupling the first magnet and the second magnet together so that the first flexible catheter and the second flexible catheter collectively surround the target tissue;
identifying, via a selection module of an electrode controller, a plurality of anode/cathode pairs, each anode selected in the plurality of anode/cathode pairs being only in the first plurality of electrodes of the first flexible catheter, each cathode selected in the plurality of anode/cathode pairs being only in the second plurality of electrodes of the second flexible catheter;
conveying a pacing signal to a pacing lead configured to be operatively coupled to the heart;
receiving, at a feedback module, an electrocardiogramal associated with a function of the heart;
delivering, via a pulse delivery module of the electrode controller, a first output signal having a first polarity to each anode selected; and
delivering, via the pulse delivery module, a second output signal having a second polarity opposite the first polarity to each cathode selected, the first output signal and the second output signal being delivered according to a sequential pattern.

2. The method of claim 1, wherein the identifying is based on an input received from an input/output module of the electrode controller.

3. The method of claim 1, further comprising:
determining an impedance between at least one anode electrode in the first plurality of electrodes and at least one cathode electrode in the second plurality of electrodes,
the identifying being performed automatically by the selection module based at least in part on the impedance.

4. The method of claim 1, further comprising:
determining the sequential pattern based on at least one of an impedance associated with the plurality of anode/cathode pairs, a distance between the plurality of anode/cathode pairs, and a characteristic associated with the heart.

5. The method of claim 1, wherein the first flexible catheter is electrically isolated from the second flexible catheter.

6. The method of claim 1, wherein the portion of the heart includes the pulmonary veins.

7. The method of claim 1, wherein an amplitude of each of the first output signal and the second output signal is up to 5 kV.

8. The method of claim 1, wherein:
an amplitude of each of the first output signal and the second output signal is up to 5 kV; and
the first flexible catheter and the second flexible catheter each includes a plurality of leads, each lead of the first flexible catheter coupled to an electrode from the first plurality of electrodes, and each lead of the second flexible catheter coupled to an electrode of the second plurality of electrodes, each lead including an outer insulating layer having a thickness from about 0.02 mm to about 0.06 mm.

9. The method of claim 1, wherein:
an amplitude of each of the first output signal and the second output signal is up to 5 kV;
the first plurality of electrodes includes at least four electrodes; and
the second plurality of electrodes includes at least four electrodes.

10. The method of claim 1, wherein:
an amplitude of each of the first output signal and the second output signal is up to 5 kV; and
the first flexible catheter and the second flexible catheter each includes a plurality of leads, each lead of the first flexible catheter coupled to an electrode from the first plurality of electrodes, and each lead of the second flexible catheter coupled to an electrode of the second plurality of electrodes, the first flexible catheter and the second flexible catheter each having a diameter of less than about 3 mm.

11. A method, comprising:
identifying, via a selection module of an electrode controller, a plurality of anode cathode pairs, each anode selected in the plurality of anode/cathode pairs being only in a first plurality of electrodes of a first multi-electrode catheter disposed about a first portion of a heart, each cathode selected in the plurality of anode/cathode pairs being only in a second plurality of electrodes of a second multi-electrode catheter disposed about a second portion of the heart, the first multi-electrode catheter including a first distal end portion having a first magnet and the second multi-electrode catheter including a second distal end portion having a second magnetic magnetically coupled to the first magnet such that the first multi-electrode catheter and the second multi-electrode catheter collectively surround a portion of a heart;
conveying a pacing signal to a pacing lead configured to be operatively coupled to the heart;
receiving, at a feedback module, an electrocardiogram signal associated with a function of the heart;
delivering, via a pulse delivery module of the electrode controller, a first output signal having a first polarity to each anode selected; and
delivering, via the pulse delivery, module, a second output signal having a second polarity opposite the first polarity to each cathode selected, the first output signal and the second output signal being delivered according to a sequential pattern.

12. The method of claim 11, wherein the identifying is based on an input received from an input/output module of the electrode controller.

13. The method of claim 11, further comprising:
determining an impedance between at least one anode electrode in the first plurality of electrodes and at least one cathode electrode in the second plurality of electrodes,
the identifying being performed automatically by the selection module based at least in part on the impedance.

14. The method of claim 11, further comprising: determining the sequential pattern based on at least one of an impedance associated with the plurality of anode/cathode pairs, a distance between the plurality of anode/cathode pairs, and a characteristic associated with the heart.

15. The method of claim 11, wherein the first multi-electrode catheter is electrically isolated from the second multi-electrode catheter.

16. The method of claim 11, wherein the portion of the heart includes the pulmonary veins.

17. The method of claim 11, wherein an amplitude of each of the first output signal and the second output signal is up to 5 kV.

18. The method of claim 11, wherein:
an amplitude of each of the first output signal and the second output signal is up to 5 kV; and
the first multi-electrode catheter and the second multi-electrode catheter each includes a plurality of leads, each lead of the first multi-electrode catheter coupled to an electrode from the first plurality of electrodes, and each lead of the second multi-electrode catheter coupled to an electrode of the second plurality of electrodes, each lead including an outer insulating layer having a thickness from about 0.02 mm to about 0.06 mm.

19. The method of claim 11, wherein:
an amplitude of each of the first output signal and the second output signal is up to 5 kV;
the first plurality of electrodes includes at least four electrodes; and
the second plurality of electrodes includes at least four electrodes.

20. The method of claim 11, wherein:
an amplitude of each of the first output signal and the second output signal is up to 5 kV; and
the first multi-electrode catheter and the second multi-electrode catheter each includes a plurality of leads, each lead of the first multi-electrode catheter coupled to an electrode from the first plurality of electrodes, and each lead of the second multi-electrode catheter coupled to an electrode of the second plurality of electrodes, the first flexible catheter and the second flexible catheter each having a diameter of less than about 3 mm.
